(19) United States
(12) Patent Application Publication
(10) Pub. No.: US 2018/0243192 A1
(43) Pub. Date: Aug. 30, 2018

(54) MOLDABLE HAIR CARE COMPOSITION

(71) Applicant: AMOREPACIFIC CORPORATION, Seoul (KR)

(72) Inventors: Yoon Kyun HWANG, Yongin-si, Gyeonggi-do (KR); Sung Ah BIN, Yongin-si, Gyeonggi-do (KR); Minjung HONG, Yongin-si, Gyeonggi-do (KR); Kyungmi LEE, Yongin-si, Gyeonggi-do (KR); Jang Won CHOI, Yongin-si, Gyeonggi-do (KR)

(73) Assignee: AMOREPACIFIC CORPORATION, Seoul (KR)

(21) Appl. No.: 15/758,523

(22) PCT Filed: Sep. 20, 2016
(86) PCT No.: PCT/KR2016/010486

§ 371 Date: Mar. 8, 2018

(30) Foreign Application Priority Data
Sep. 30, 2015 (KR) 10-2015-0137535

(51) Int. Cl. A61K 8/41; A61K 8/02; A61K 8/81; A61K 8/46; A61K 8/42; A61K 8/34; A61Q 5/02; A61Q 5/12; B65D 75/36

(52) U.S. Cl. CPC A61K 8/416; A61K 8/0216; A61K 8/8147; A61K 8/463; A61K 2800/87; A61K 8/342; A61Q 5/02; A61Q 5/12; B65D 75/36; A61K 8/42

(57) ABSTRACT

The present specification relates to a moldable solid preparation hair care composition containing a cationic surfactant, an anionic surfactant and a thickener, wherein the amount of cationic surfactant is 0.1-5 wt % on the basis of the total composition, the amount of anionic surfactant is greater than or equal to 0.1 wt % and less than 1 wt % on the basis of the total composition, and the amount of thickener is 0.1-15 wt % on the basis of the total composition. According to one aspect of the present invention, the hair care composition has excellent moldability as a solid preparation and, simultaneously, exhibits remarkable conditioning effects.

### TECHNICAL FIELD

The present disclosure relates to a shapable solid composition for hair care, which includes a cationic surfactant, anionic surfactant and a viscosity increasing agent.

### BACKGROUND ART

The hair of adults may be damaged severely by contamination, frequent permanent, hair drying, hair dyeing and combing. Therefore, a hair cleaning agent having at least a predetermined level of conditioning effect is required. Conventional hair care products for a conditioning effect are provided in the type of a liquid or viscous material and are used by taking such a liquid or viscous material on hands. In other words, such conventional formulations are creamy emulsion formulations, and thus the products cannot be shaped and packed in a desired shape or in an individually wrapped shape.

Meanwhile, a hair care composition for a conditioning effect is required during camping or travelling in addition to the use in daily life. Thus, it is required for a hair care product to have convenient portability.

Therefore, there is a need for studying a solid composition for hair care having shaping convenience and shaping stability so that it may be applied as a commercial product convenient to use during camping or travelling, while not causing degradation of a conditioning effect proper to a hair care product.

### REFERENCES

### Patent Documents

Korean Patent Laid-Open No. 10-2011-0026525

### DESCRIPTION OF DRAWINGS

FIG. 1 and FIG. 2 are photographs of the shapable solid compositions having hexahedral, tetrahedral and spherical shapes according to the embodiments of the present disclosure.

### DISCLOSURE

### Technical Problem

A technical problem to be solved by the present disclosure is to provide a shapable solid composition for hair care, which includes a cationic surfactant, anionic surfactant and a viscosity increasing agent.

Another technical problem to be solved by the present disclosure is to provide a shapable solid composition for hair care also having an excellent conditioning effect.

Still another technical problem to be solved by the present disclosure is to provide a shapable solid composition for hair care, wherein the viscosity increasing agent is a polyacrylate salt.

Still another technical problem to be solved by the present disclosure is to provide a shapable solid composition for hair care, wherein the cationic surfactant is at least one cationic surfactant selected from behentrimonium chloride, steartrimonium chloride, behentrimonium methosulfate and stearamidopropyldimethyl amine; the anionic surfactant is at least one anionic surfactant selected from disodium laureth sulfosuccinate, sodium lauryl glucose carboxylate, disodium lauryl sulfosuccinate, sodium polyoxyethylene lauryl ether sulfate, sodium C14-C16 olefin sulfonate and ammonium laureth sulfate; and the viscosity increasing agent is at least one viscosity increasing agent selected from sodium polyacrylate and sodium polyacrylate starch.

Still another technical problem to be solved by the present disclosure is to provide a shapable solid composition for hair care, which further includes a higher aliphatic alcohol having 12 or more carbon atoms.

Still another technical problem to be solved by the present disclosure is to provide a shapable solid composition for hair care, which is a leave-on type or wash-off type composition.

Yet another technical problem to be solved by the present disclosure is to provide a hair care product including a shaped hair care material obtained from the composition for hair care and a blister packaging, wherein the shaped hair care material is present as a solid formulation in the blister packaging without coating.

### Technical Solution

In one general aspect, there is provided a shapable solid composition for hair care, which includes a cationic surfactant, anionic surfactant and a viscosity increasing agent.

According to an embodiment, there is provided a shapable solid composition for hair care, wherein the viscosity increasing agent is a polyacrylate salt.

According to another embodiment, there is provided a shapable solid composition for hair care, wherein the polyacrylate salt has a weight average molecular weight of 1,000-85,000 Mw.

According to still another embodiment, there is provided a shapable solid composition for hair care, wherein the cationic surfactant is at least one cationic surfactant selected from behentrimonium chloride, steartrimonium chloride, behentrimonium methosulfate and stearamidopropyldimethyl amine; the anionic surfactant is at least one anionic surfactant selected from disodium laureth sulfosuccinate, sodium lauryl glucose carboxylate, disodium lauryl sulfosuccinate, sodium polyoxyethylene lauryl ether sulfate, sodium C14-C16 olefin sulfonate and ammonium laureth sulfate; and the viscosity increasing agent is at least one viscosity increasing agent selected from sodium polyacrylate and sodium polyacrylate starch.

According to still another embodiment, there is provided a shapable solid composition for hair care, wherein the cationic surfactant is used in an amount of 0.1-5 wt % based on the total weight of the composition, the anionic surfactant is used in an amount of 0.1 wt % or more and less than 1 wt % based on the total weight of the composition, and the viscosity increasing agent is used in an amount of 0.1-15 wt % based on the total weight of the composition.

According to still another embodiment, there is provided a shapable solid composition for hair care, wherein the cationic surfactant, anionic surfactant and the viscosity increasing agent are used at a weight ratio of 1:0.1-1:0.5-12. Particularly, the cationic surfactant, anionic surfactant and the viscosity increasing agent are used at a weight ratio of 1:0.2-0.6:0.5-5.

According to still another embodiment, there is provided a shapable solid composition for hair care, which further includes a higher aliphatic alcohol having 12 or more carbon atoms. Particularly, the higher aliphatic alcohol may be at least one selected from cetyl alcohol, stearyl alcohol and cetearyl alcohol.

According to still another embodiment, there is provided a shapable solid composition for hair care, wherein the higher aliphatic alcohol and viscosity increasing agent are used at a weight ratio of 10-1:1-10.

According to still another embodiment, there is provided a shapable solid composition for hair care, which further includes a pH adjusting agent.

According to still another embodiment, there is provided a shapable solid composition for hair care, which is a leave-on type or wash-off type composition.

According to yet another embodiment, there is provided a hair care product including a shaped hair care material obtained from the composition for hair care and a blister packaging, wherein the shaped hair care material is present as a solid formulation in the blister packaging without coating.

### Advantageous Effects

The composition according to an embodiment of the present disclosure is a solid formulation having excellent solid stability.

The composition according to an embodiment of the present disclosure is a solid formulation having excellent shapability.

The composition according to an embodiment of the present disclosure is a solid formulation having excellent shear (force) resistance.

The composition according to an embodiment of the present disclosure is a solid formulation that may be provided with various textures.

The composition according to an embodiment of the present disclosure shows an excellent treatment effect while providing a solid formulation, when it is applied to hair.

The composition according to an embodiment of the present disclosure shows an excellent conditioning effect while providing a solid formulation, when it is applied to hair.

The composition according to an embodiment of the present disclosure shows excellent softness and smoothness, when it is applied to hair.

The composition according to an embodiment of the present disclosure shows excellent spreadability, when it is applied to hair.

The composition according to an embodiment of the present disclosure may provide a leave-on type hair care composition.

The composition according to an embodiment of the present disclosure may provide a wash-off type hair care composition.

The hair care composition including the composition according to an embodiment of the present disclosure may be provided as a blister packaged type product without coating.

### BEST MODE

Exemplary embodiments now will be described more fully hereinafter so that the present disclosure will fully convey the scope of the present disclosure to those skilled in the art.

In one aspect, there is provided a shapable solid composition for hair care, which includes a cationic surfactant, anionic surfactant and a viscosity increasing agent.

In another aspect, there is provided use of a cationic surfactant, anionic surfactant and a viscosity increasing agent for preparing a shapable solid composition for hair care.

In still another aspect, there is provided a method for hair care of a subject, which includes a step of applying a shapable solid composition for hair care, including a cationic surfactant, anionic surfactant and a viscosity increasing agent, to the subject.

As used herein, the term ‘shapable solid’ means a solid formulation which is easily shaped into a specific shape, such as a spherical shape, tetrahedral shape or hexahedral shape, by hands or by using a machine and is not broken under a predetermined level of force (shear) after shaping.

According to an embodiment of the shapable solid composition for hair care, the viscosity increasing agent is a polyacrylate salt.

According to another embodiment of the shapable solid composition for hair care, the polyacrylate salt has a weight average molecular weight of 1,000-85,000 Mw.

According to a particular embodiment of the shapable solid composition for hair care, the polyacrylate salt may have a molecular weight of at least 1,000 Mw, at least 1,200 Mw, at least 1,500 Mw, at least 2,000 Mw, at least 5,000 Mw, at least 10,000 Mw, at least 15,000 Mw, at least 30,000 Mw or at least 40,000 Mw.

In addition, the polyacrylate salt may have a molecular weight of at most 85,000 Mw, at most 83,000 Mw, at most 80,000 Mw, at most 75,000 Mw, at most 70,000 Mw, at most 65,000 Mw, at most 60,000 Mw, at most 50,000 Mw, or at most 45,000 Mw.

When the polyacrylate salt has a molecular weight within the above-defined range, it is possible to provide a solid composition having an excellent conditioning effect and to provide a viscosity with which the composition can be shaped into a specific shape.

According to still another embodiment of the shapable solid composition for hair care, the cationic surfactant is at least one cationic surfactant selected from behentrimonium chloride, steartrimonium chloride, behentrimonium methosulfate and stearamidopropyl dimethyl amine. According to an embodiment, when using various cationic surfactants, the solid composition may be shaped with ease. According to another embodiment, when using stearamidopropyl dimethyl amine is used as a cationic surfactant, smooth dissolution may not be accomplished during the preparation of the composition. Thus, a pH adjusting agent, such as lactic acid, may be used to modify pH and accomplish dissolution. According to still another embodiment, when using stearamidopropyl dimethyl amine as a cationic surfactant, the resultant composition has a lower viscosity as compared to the composition using a different cationic surfactant. Therefore, the viscosity increasing agent may be used in an increased amount as compared to the composition using a different cationic surfactant so that the composition may have a desired level of viscosity. According to still another embodiment, when using a combination of the above surfactants, the solid composition may be shaped with ease.

According to still another embodiment of the shapable solid composition for hair care, the anionic surfactant is at least one anionic surfactant selected from disodium laureth sulfosuccinate, sodium lauryl glucose carboxylate, disodium lauryl sulfosuccinate, sodium polyoxyethylene lauryl ether sulfate, sodium C14-C16 olefin sulfonate and ammonium laureth sulfate, and the viscosity increasing agent is at least one viscosity increasing agent selected from sodium polyacrylate and sodium polyacrylate starch. According to an embodiment, similarly to the use of the cationic surfactant, use of various anionic surfactants allows easy shaping into a solid composition.

According to still another embodiment of the shapable solid composition for hair care, the viscosity increasing agent is at least one selected from sodium polyacrylate and sodium polyacrylate starch.

According to still another embodiment of the shapable solid composition for hair care, the cationic surfactant is used in an amount of 0.1-5 wt % based on the total weight of the composition, the anionic surfactant is used in an amount of 0.1 wt % or more and less than 1 wt % based on the total weight of the composition, and the viscosity increasing agent is used in an amount of 0.1-15 wt % based on the total weight of the composition.

Particularly, the cationic surfactant may be used in an amount of at least 0.1 wt %, at least 0.2 wt %, at least 0.3 wt %, at least 0.4 wt %, at least 0.5 wt %, at least 0.6 wt %, at least 0.7 wt %, at least 0.8 wt %, at least 0.9 wt %, at least 1.0 wt %, at least 1.1 wt %, at least 1.2 wt %, at least 1.3 wt %, at least 1.4 wt %, at least 1.5 wt %, at least 2.0 wt % or at least 3.0 wt % based on the total weight of the composition.

In addition, the cationic surfactant may be used in an amount of at most 5.0 wt %, at most 4.0 wt %, at most 3.0 wt %, at most 2.0 wt %, at most 1.9 wt %, at most 1.8 wt %, at most 1.7 wt %, at most 1.6 wt %, at most 1.5 wt %, at most 1.4 wt %, at most 1.3 wt %, at most 1.2 wt %, at most 1.1 wt %, at most 1.0 wt %, at most 0.9 wt %, at most 0.8 wt %, at most 0.7 wt % or at most 0.6 wt % based on the total weight of the composition.

When the cationic surfactant is used within the above-defined range, it is possible to provide a solid composition having an excellent conditioning effect and to provide a viscosity with which the composition can be shaped into a specific shape.

Particularly, the anionic surfactant may be used in an amount of at least 0.1 wt %, at least 0.2 wt %, at least 0.3 wt %, at least 0.4 wt %, at least 0.5 wt %, at least 0.6 wt % or at least 0.7 wt % based on the total weight of the composition.

In addition, the anionic surfactant may be used in an amount less than 1.0 wt %, at most 0.9 wt %, at most 0.8 wt %, at most 0.7 wt % or at most 0.6 wt % based on the total weight of the composition.

When the anionic surfactant is used within the above-defined range, it is possible to provide a solid composition having an excellent conditioning effect and to provide a viscosity with which the composition can be shaped into a specific shape.

Particularly, the viscosity increasing agent may be used in an amount of at least 0.1 wt %, at least 0.3 wt %, at least 0.5 wt %, at least 0.8 wt %, at least 0.9 wt %, at least 1.0 wt %, at least 1.1 wt %, at least 1.2 wt %, at least 1.3 wt %, at least 1.4 wt %, at least 1.5 wt %, at least 2.0 wt %, at least 3.0 wt %, at least 4.0 wt %, at least 5.0 wt %, at least 7.0 wt % or at least 9.0 wt % based on the total weight of the composition.

In addition, the viscosity increasing agent may be used in an amount of at most 15 wt %, at most 13 wt %, at most 11 wt %, at most 10 wt %, at most 8 wt %, at most 5 wt %, at most 3 wt %, at most 2 wt %, at most 1.8 wt %, at most 1.5 wt %, at most 1.4 wt %, at most 1.3 wt %, at most 1.2 wt %, at most 1.1 wt % or at most 1.0 wt % based on the total weight of the composition.

When the viscosity increasing agent is used within the above-defined range, it is possible to provide a solid composition having an excellent conditioning effect and to provide a viscosity with which the composition can be shaped into a specific shape.

According to still another embodiment of the shapable solid composition for hair care, the cationic surfactant, anionic surfactant and the viscosity increasing agent are used at a weight ratio of 1:0.1-1:0.5-12. Particularly, the cationic surfactant, anionic surfactant and the viscosity increasing agent are used at a weight ratio of 1:0.2-0.6:0.5-5. More particularly, cationic surfactant, anionic surfactant and the viscosity increasing agent are used at a weight ratio of 1:0.3:1-2. As used herein, the range of weight ratio includes any numbers present between the two terminal values. Therefore, for example, the weight ratio of 1:0.3:1-2 may cover a weight ratio of 1:0.3:1.1, 1:0.3:1.2, 1:0.3:1.3, 1:0.3:1.4, 1:0.3:1.5, 1:0.3:1.6, 1:0.3:1.7, 1:0.3:1.8 or 1:0.3:1.9, but is not limited thereto, as generally known to those skilled in the art.

According to still another embodiment of the shapable solid composition for hair care, the composition includes a higher aliphatic alcohol having 12 or more carbon atoms. Particularly, the higher aliphatic alcohol may be at least one selected from cetyl alcohol, stearyl alcohol and cetearyl alcohol, but is not limited thereto.

According to still another embodiment of the shapable solid composition for hair care, the higher aliphatic alcohol and viscosity increasing agent are used at a weight ratio of 10-1:1-10.

According to still another embodiment of the shapable solid composition for hair care, the composition further includes a pH adjusting agent. Particularly, the pH adjusting agent may include lactic acid, but is not limited thereto.

According to yet another embodiment of the shapable solid composition for hair care, the composition is a leave-on type or wash-off type hair care composition.

In yet another aspect, there is provided a hair care product including a shaped hair care material obtained from the composition for hair care and a blister packaging, wherein the shaped hair care material is present as a solid formulation in the blister packaging without coating.

Hereinafter, the present disclosure will be explained in detail with reference to examples and comparative examples. However, the following examples are for illustrative purposes only and not intended to limit the scope of this disclosure. It will be understood by those skilled in the art that modifications, substitutions and insertions may be made to the following examples and test examples without departing from the spirit and scope of the present disclosure as defined by the appended claims.

### Examples 1-15 and Comparative Example

The compositions for hair care according to Examples 1-15 and Comparative Example are obtained according to the compositions as shown in the following Table 1. The following ingredients used for the compositions are commercially available products.

Behentrimonium chloride: Miwon Commercial Co., Ltd.

Steartrimonium chloride: Miwon Commercial Co., Ltd.

Behentrimonium methosulfate*Cetyl alcohol*Butylene glycol: CRODA INC.

Stearamidopropyl dimethyl amine: Miwon Commercial Co., Ltd.

Disodium laureth sulfosuccinate: Miwon Commercial Co., Ltd.

Sodium polyacrylate: Sigma Aldrich (Mw: 1,200-83,000)

Sodium polyacrylate starch: DAITO KASEI KOGYO CO., LTD

TABLE 1

| | Comp. | | | | | | | |
|---|---|---|---|---|---|---|---|---|
| | Ex. | Ex. 1 | Ex. 2 | Ex. 3 | Ex. 4 | Ex. 5 | Ex. 6 | Ex. 7 |
| Purified water | To 100 | To 100 | To 100 | To 100 | To 100 | To 100 | To 100 | To 100 |
| Behentrimonium | 1 | 1 | — | — | — | 0.5 | 0.5 | 0.5 |
| chloride | | | | | | | | |
| Steartrimonium | — | — | 1 | — | — | 0.5 | — | — |
| chloride | | | | | | | | |
| Behentrimonium | — | — | — | 1 | — | — | 0.5 | — |
| methosulfate * cetyl | | | | | | | | |
| alcohol * butylene | | | | | | | | |
| glycol | | | | | | | | |
| Stearamidopropyl | — | — | — | — | 1 | — | — | 0.5 |
| dimethyl amine | | | | | | | | |
| Lactic acid | — | — | — | — | 0.3 | — | — | 0.15 |
| Disodium laureth | — | 0.3 | 0.3 | 0.3 | 0.3 | 0.3 | 0.3 | 0.3 |
| sulfosuccinate | | | | | | | | |
| Cetearyl alcohol | 8 | 8 | 8 | 8 | 8 | 8 | 8 | 8 |
| Stearyl alcohol | — | — | — | — | — | — | — | — |
| Cetyl alcohol | — | — | — | — | — | — | — | — |
| Hydrogenated | 5 | 5 | 5 | 5 | 5 | 5 | 5 | 5 |
| Polyisobutene | | | | | | | | |
| Glycerin | 15 | 15 | 15 | 15 | 15 | 15 | 15 | 15 |
| Dimethicone | 2 | 2 | 2 | 2 | 2 | 2 | 2 | 2 |
| Phenoxyethanol | 0.3 | 0.3 | 0.3 | 0.3 | 0.3 | 0.3 | 0.3 | 0.3 |
| Fragrance | 0.3 | 0.3 | 0.3 | 0.3 | 0.3 | 0.3 | 0.3 | 0.3 |
| Sodium | — | 0.5 | 0.5 | 0.5 | 0.7 | 0.5 | 0.5 | 0.6 |
| Polyacrylate | | | | | | | | |
| Sodium | — | 0.5 | 0.5 | 0.5 | 0.7 | 0.5 | 0.5 | 0.6 |
| Polyacrylate Starch | | | | | | | | |
| | E. | | Ex. | Ex. | Ex. | Ex. | Ex. | Ex. |
| | x8 | Ex. 9 | 10 | 11 | 12 | 13 | 14 | 15 |
| Purified water | To 100 | To 100 | To 100 | To 100 | To 100 | To 100 | To 100 | To 100 |
| Behentrimonium | 1 | 1 | 1 | 1 | 1 | 1 | 1 | 1 |
| chloride | | | | | | | | |
| Steartrimonium | — | — | — | — | — | — | — | — |
| chloride | | | | | | | | |
| Behentrimonium | — | — | — | — | — | — | — | — |
| methosulfate * cetyl | | | | | | | | |
| alcohol * butylene | | | | | | | | |
| glycol | | | | | | | | |
| Stearamidopropyl | — | — | — | — | — | — | — | — |
| dimethyl amine | | | | | | | | |
| Lactic acid | — | — | — | — | — | — | — | — |
| Disodium laureth | 0.3 | 0.3 | 0.3 | 0.3 | 0.3 | — | 0.5 | 1 |
| sulfosuccinate | | | | | | | | |
| Cetearyl alcohol | — | — | 5 | 8 | 3 | 8 | 8 | 8 |
| Stearyl alcohol | 6 | 4 | — | — | — | — | — | — |
| Cetyl alcohol | 2 | 4 | — | — | — | — | — | — |
| Hydrogenated | 5 | 5 | 5 | 5 | 5 | 5 | 5 | 5 |
| Polyisobutene | | | | | | | | |
| Glycerin | 15 | 15 | 15 | 15 | 15 | 15 | 15 | 15 |
| Dimethicone | 2 | 2 | 2 | 2 | 2 | 2 | 2 | 2 |
| Phenoxyethanol | 0.3 | 0.3 | 0.3 | 0.3 | 0.3 | 0.3 | 0.3 | 0.3 |
| Fragrance | 0.3 | 0.3 | 0.3 | 0.3 | 0.3 | 0.3 | 0.3 | 0.3 |
| Sodium | 0.5 | 1 | 2 | 2 | 5 | 0.5 | 0.5 | 0.5 |
| Polyacrylate | | | | | | | | |
| Sodium | 0.7 | 1 | 2 | 2 | 5 | 0.5 | 0.5 | 0.5 |
| Polyacrylate Starch | | | | | | | | |

### [Test Example] Test for Solid Formulation Convertibility and Shapability of Composition

Examples 1-15 are determined for solid formulation convertibility and shapability. The ingredients of Comparative Example are base ingredients of the conventional treatment or rinse and cannot be shaped into a spherical shape even though the content of surfactant or higher aliphatic alcohol is increased.

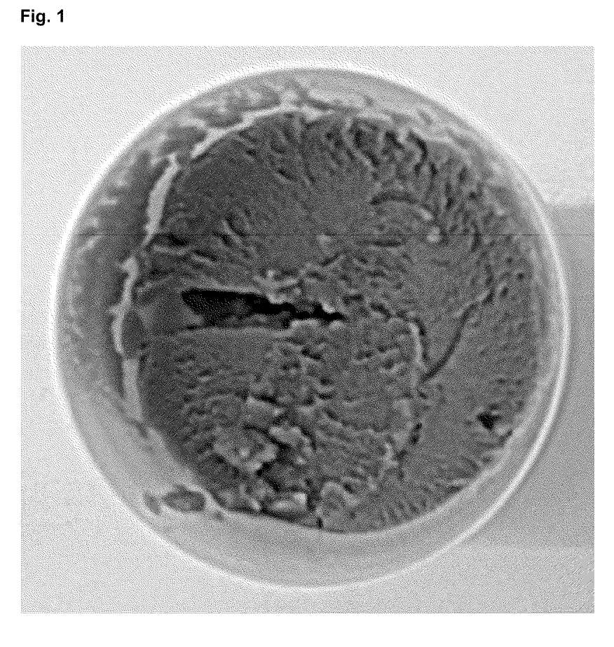

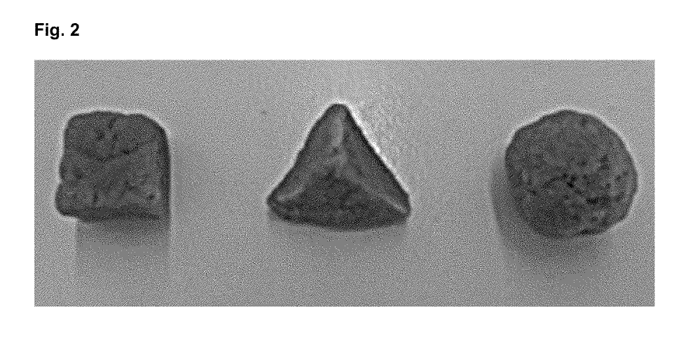

On the contrary, Examples 1-15 are provided as solid formulations as shown in FIG. 1. Particularly, the shape of each of the solid compositions according to Examples 1-14 demonstrates easy shapability and shaping stability as shown in FIG. 2.

The compositions according to Examples 1-4 demonstrates that even when using a different type of cationic surfactant, the compositions can be shaped into a spherical shape.

The compositions according to Examples 5-7 demonstrates that when using at least one type of cationic surfactant, various combinations of cationic surfactants allows shaping into a spherical shape.

The compositions further including a higher aliphatic alcohol according to Examples 8-9 also demonstrates shapability into a spherical shape.

In addition, Examples 10-14 using differently controlled amounts of viscosity increasing agent and anionic surfactant demonstrates shapability into a spherical shape. However, in the case of Example 15, when the composition is shaped into a spherical shape, it shows insufficient stability.

### [Test Example 2] Test for Conditioning Effect—Softness and Spreadability of Compositions

To determine the softness and spreadability of the composition according to the present disclosure upon application to hair, Examples 1-14 and Comparative Example are tested as follows.

The spreadability on hands when using each composition and the softness/smoothness of each composition upon application to hair are evaluated by 20 professional panels. After using each of the compositions according to Comparative Example and Examples 1-14, the test is carried out and each composition is evaluated by giving a point of 1 to the lowest result and a point of 5 to the highest result. The results are shown in the following Table 2 as average values.

TABLE 2

| | Comp. | | | | | | | | | | Ex. | Ex. | Ex. | Ex. | Ex. |
|---|---|---|---|---|---|---|---|---|---|---|---|---|---|---|---|
| | Ex. | Ex. 1 | Ex. 2 | Ex. 3 | Ex. 4 | Ex. 5 | Ex. 6 | Ex. 7 | Ex. 8 | Ex. 9 | 10 | 11 | 12 | 13 | 14 |
| Softness/ | 4.3 | 4.2 | 4.3 | 4.2 | 4.5 | 4.3 | 4.2 | 4.5 | 4 | 4.5 | 3.5 | 3.8 | 3.2 | 4.2 | 3.8 |
| smoothness | | | | | | | | | | | | | | | |
| Spreadability | 4.6 | 4.5 | 4.6 | 4.4 | 4.5 | 4.5 | 4.5 | 4.6 | 4.3 | 4.4 | 4 | 3.8 | 3.3 | 4.5 | 4.1 |

After the test, it can be seen that Examples 1-14 provide a level of conditioning effect similar to that of Comparative Example, i.e., have excellent spreadability and softness.

1. A shapable solid composition for hair care, which comprises a cationic surfactant, anionic surfactant and a viscosity increasing agent.

2. The shapable solid composition for hair care according to claim 1, wherein the viscosity increasing agent is a polyacrylate salt.

3. The shapable solid composition for hair care according to claim 2, wherein the polyacrylate salt has a weight average molecular weight of 1,000-85,000 Mw.

4. The shapable solid composition for hair care according to claim 1, wherein
the cationic surfactant is at least one cationic surfactant selected from behentrimonium chloride, steartrimonium chloride, behentrimonium methosulfate and stearamidopropyldimethyl amine;
the anionic surfactant is at least one anionic surfactant selected from disodium laureth sulfosuccinate, sodium lauryl glucose carboxylate, disodium lauryl sulfosuccinate, sodium polyoxyethylene lauryl ether sulfate, sodium C14-C16 olefin sulfonate and ammonium laureth sulfate; and
the viscosity increasing agent is at least one viscosity increasing agent selected from sodium polyacrylate and sodium polyacrylate starch.

5. The shapable solid composition for hair care according to claim 1, wherein the cationic surfactant is used in an amount of 0.1-5 wt % based on the total weight of the composition, the anionic surfactant is used in an amount of 0.1 wt % or more and less than 1 wt % based on the total weight of the composition, and the viscosity increasing agent is used in an amount of 0.1-15 wt % based on the total weight of the composition.

6. The shapable solid composition for hair care according to claim 1, wherein the cationic surfactant, anionic surfactant and the viscosity increasing agent are used at a weight ratio of 1:0.1-1:0.5-12.

7. The shapable solid composition for hair care according to claim 1, wherein the cationic surfactant, anionic surfactant and the viscosity increasing agent are used at a weight ratio of 1:0.2-0.6:0.5-5.

8. The shapable solid composition for hair care according to claim 1, which further comprises a higher aliphatic alcohol having 12 or more carbon atoms.

9. The shapable solid composition for hair care according to claim 8, wherein the higher aliphatic alcohol is at least one selected from cetyl alcohol, stearyl alcohol and cetearyl alcohol.

10. The shapable solid composition for hair care according to claim 8, wherein the higher aliphatic alcohol and viscosity increasing agent are used at a weight ratio of 10-1:1-10.

11. The shapable solid composition for hair care according to claim 1, which further comprises a pH adjusting agent.

12. The shapable solid composition for hair care according to claim 1, which is a leave-on type or wash-off type composition.

13. A hair care product comprising a shaped hair care material obtained from the composition for hair care as defined in claim 1 and a blister packaging, wherein the shaped hair care material is present as a solid formulation in the blister packaging without coating.
